(19) United States
(12) Patent Application Publication
(10) Pub. No.: US 2017/0014822 A1
(43) Pub. Date: Jan. 19, 2017

(54) MICROFLUIDIC CARTRIDGE AND READER DEVICE, SYSTEM, AND METHOD OF USE

(71) Applicant: Aark Health Private Limited, Singapore (SG)

(72) Inventor: Justin R. Ker, SG (SG)

(73) Assignee: Aark Health Private Limited, Singapore (SG)

(21) Appl. No.: 15/211,254

(22) Filed: Jul. 15, 2016

Related U.S. Application Data
(60) Provisional application No. 62/192,644, filed on Jul. 15, 2015.

(51) Int. Cl. B01L 3/00; G01N 27/327; G01N 33/487; G01N 27/416

(52) U.S. Cl. CPC B01L 3/502715; G01N 27/4161; G01N 27/3272; G01N 27/3273; G01N 27/3275; G01N 33/48707; B01L 2300/0887; B01L 2300/0681; B01L 2300/0627; B01L 2300/0645; B01L 2300/021; B01L 2300/023

(57) ABSTRACT

Disposable microfluidic cartridges, potentiostat reader devices, systems, kits, and methods to determine the concentration of analyte reaction products in a patient's specimen, such as: blood, saliva, cerebrospinal fluid, joint fluid or tears. The microfluidic cartridges are disposable, paper-based strips comprising multiple assay layers, each layer comprising: a drop zone for receiving a patient specimen; a filter to remove contaminates, and/or isolate assay reactants; a hydrophilic microfluidic channel to direct movement of the specimen down the cartridge; a centered reaction chamber comprising impregnated enzymes and reagents to chemically react with the filtered specimen to produce an analyte; and a detector mechanism. Multiple analytes can be produced in parallel (e.g. one per layer), or sequentially along one layer. The detector mechanism utilizes electro-analytic methods to facilitate the reader device in quantifying each analyte, and each analyte concentration is wirelessly transmitted to an electronic computing device for storage in a patient's medical record.

### PRIORITY CLAIM

This application claims priority to Provisional U.S. Patent Application Ser. No. 62/192,644 filed Jul. 15, 2015, entitled “MICROFLUIDIC CARTRIDGE AND READER DEVICE, SYSTEM, METHOD OF USE”, and which is hereby incorporated by reference in its entirety.

### FIELD OF THE DISCLOSURE

The present disclosure generally relates to disposable microfluidic devices comprising cartridges (i.e. sensor strips) for point-of-care medical use to quickly analyze the concentration of an analyte in a patient's specimen, such as blood. The cartridges are inserted into a potentiostat reader device that calculates the amount or concentration of one or more analytes in the specimen.

### COPYRIGHT NOTICE

A portion of the disclosure of this patent application document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### TRADEMARKS DISCLAIMER

The product names used in this document are for identification purposes only. All trademarks and registered trademarks are the property of their respective owners.

### BACKGROUND OF THE DISCLOSURE

In medical diagnostics, a physician will often order laboratory tests run on a patient's bodily fluid specimen, such as blood, saliva, etc., in order to determine the absence or presence of an analyte that is used to diagnose a patient's health. The patient must often travel to another medical facility location to have their specimen taken, and many patients will not expend the time and energy to have the tests run. Or, the physician may take the specimen, and have it transported to an outside laboratory for testing, which results in a time delay in getting the test results. Therefore, patient compliance and health can be significantly improved with a point-of-care (POC) system that allows a physician to efficiently test a patient's specimen within the office setting, and to immediately receive the test results.

Microfluidic devices are point-of-care (POC) medical testing devices that require the application of a patient's fluid specimen (e.g. blood) onto a cartridge (e.g. test sensor strip), that is inserted to an electrical reader device. The device then computes and displays the amount or concentration of an analyte in the specimen. A common example is blood glucose devices that analyze a drop of a diabetic's blood to compute and display the concentration of glucose in the blood. What these devices currently lack, though, is the ability to run more complicated diagnostic tests, such as testing for lipids, cholesterol, hemoglobin, liver enzymes, DNA, RNA, and proteins (e.g. antibodies) associated with a specific disease or disorder.

What is also lacking within point-of-care diagnostic devices, is a system that: 1) does not require centrifugation or other labor and time consuming clinician steps; 2) uses disposable cartridge-sensor strips to prevent specimen contamination and false test results; and, 3) automatedly enters the test results into a patient's electronic medical records, including those stored on a doctor's computer servers, and/or a patient's mobile device.

### Microfluidic Devices

Cyclic voltammetry (CV) and cyclic amperometry (CA) are two commonly used electro-analytical methods in many areas of chemistry. They are often used to determine the stability of reaction products, the presence of intermediates in redox reactions, and electron-transfer kinetics. These methods are currently used in cellular biology for measuring bio-samples.

A potentiostat is the electronic hardware required to control three electrode cells and run these electro-analytical experiments. However, commercial available potentiostats are bulky and expensive, which limit their usage to professional laboratory experiments. For example, a wireless version of the i-STAT® from Abbott Labs costs about $20,600; and, the Piccolo Xpress® by Abaxis system offers 31 blood chemistry tests for about $18-25,000.

What is needed within the point-of-care (POC) industry, is a low cost and portable electrochemical workstation for home or clinic usage. With the addition of Wi-Fi capability, it should be able to be implemented as an Internet of Things (IOT) device, where the experiments can be controlled and monitored remotely through the internet, and the results can be stored in the Cloud and be accessed by mobile device Apps.

### SUMMARY OF THE DISCLOSURE

The present disclosure comprises a microfluidic system using cyclic voltammetry for expeditiously detecting and quantifying at least one analyte within a patient's specimen in order to facilitate quickly and economically diagnosing a patient's medical condition. The various embodiments of the present disclosure comprise: a disposable, multi-layered microfluidic cartridge; a potentiostat reader device into which the cartridge is inserted, or otherwise connected, and analyzed using cyclic voltammetry; a computerized system and a computer program product to compute the amount or concentration of one or more analytes on the cartridge; and a method of use of the entire microfluidic system.

More specifically, the present disclosure comprises: 1) a disposable multi-layered microfluidic cartridge comprising paper and/or plastic sensor strips impregnated with enzymes and/or reagents specifically designed to produce a desired analyte reaction product (or “analyte”); and 2) an electronic potentiostat reader device able to detect and/or quantify the cartridge analyte. In an embodiment, the reader device is a hand-held potentiostat device able to perform cyclic voltammetry and/or amperometry on clinical samples (e.g. the patient filtered specimen imbedded in the microfluidic cartridge).

The present disclosure further comprises a kit comprising at least one microfluidic cartridge, and optionally a cartridge potentiostat reader device

The present disclosure further comprises a computerized microfluidic system comprising one or more of the following components: a barcode or QR code reader device (e.g. smartphone with a QR code scanner mobile app) to correlate a patient specimen with a reader device output; at least one disposable multi-layered microfluidic cartridge device; a portable, hand-held cartridge potentiostat reader device; a wireless network; and at least one electronic computing device with the computer program product of the present disclosure installed or accessible thereon to wirelessly receive, analyze, and store the analyte results.

The various embodiments of the present disclosure comprise a disposable multi-layered microfluidic cartridge (i.e. paper sensor strips) impregnated with enzymes and reagents to isolate analytes from a patient's specimen. An analyte is the constituent of the patient specimen, or a reaction product derived from the specimen, that is detected and quantified by a reader device that the cartridge is inserted into, or otherwise connected to. The analytes (i.e. assay reaction products) may comprise, by way of non-limiting examples, one or more of: hemoglobin, platelets, sodium, potassium, creatinine, urea, lactic acid, cholesterol, low density lipoprotein, high density lipoprotein, triglycerides, testosterone, cortisol, prostate specific antigens, and tumor markers.

In an embodiment, the patient's specimen is placed near the rear end of the paper cartridge, and as it is wicked towards the cartridge front end (e.g. via capillaries within the paper layers), it is filtered to isolate specific components that then chemically react with the enzymes and reagents in the middle of the cartridge to produce one or more analyte reaction productions (i.e. “analytes”). The analyte then travels to the front end of the cartridge comprising three parallel electrodes surrounded by the analytes, which is inserted into, or otherwise connected to, a potentiostat reader device that detects and/or computes the quantity (e.g. concentration) of the analyte in the specimen.

The enzymes and reagents impregnated within each layer of a cartridge reaction chamber are therefore specifically designed to react with a selected filtered specimen component to produce a desired analyte reaction product.

In the various embodiments disclosed herein, the reader device is a portable, hand-held potentiostat device, and the front end of the cartridge comprises a plurality of parallel electrodes that the potentiostat device reads the electrical potential of. The concentration of the analyte is computed from the electrical potential by methods well known in the art cyclic voltammetry, such as from digital graphs of analyte concentration versus analyte current (e.g. FIGS. 9 and 10).

### Disposable Microfluidic Cartridge

Each microfluidic cartridge comprises: a thin, rectangular shaped sensor strip, enabled for use one time and then disposed of. Furthermore, the cartridges are made of disposable paper, plastic, and/or cellulose material that is able to wick a filtered specimen, and its analyte reaction product, from the rear end to the front end of the cartridge, along a center lane made of hydrophilic material.

Each cartridge may further comprise one or more vertically stacked analyte layers (e.g. see FIGS. 3-5), hence one cartridge may generate a different analyte reaction product on each layer for detection and quantification by the reader device. For example, the screen on the reader device, or a computer that the analyte data was transferred to, would sequentially or concurrently display the concentration of each analyte reaction product within the cartridge, such as from the top cartridge layer to the bottom cartridge layer.

Each cartridge layer further comprises thin, rectangular shaped components of: 1) a backing strip that provides structural support to one or more assay layers; and, 2) one or more assay layers stacked vertically upon the backing strip.

Each assay layer (2) comprises from the rear end to the front end: a drop zone for receiving a droplet of a patient specimen; a filter that is able to remove contaminates, and/or to isolate pre-selected assay reactants; a reaction chamber centered in the layer and impregnated with enzymes and/or reagents to chemically react with the filtered specimen assay reactants to produce one or more analyte reaction products comprising ions; and, a detection mechanism (e.g. three parallel electrodes surrounded by analyte) located at the cartridge front end that is able to facilitate the potentiostat reader device in detecting and quantifying the amount or concentration of the one or more analyte reaction products.

Each assay layer (2) may further comprise: a straight hydrophilic microfluidic channel extending from the filter to the rear end of the center reaction chamber, and from the front end of the reaction chamber to the detector mechanism. The filtered assay reactants are hydrophilic, and are thus able to travel (via wicking) through the hydrophilic channels, but not the outlying hydrophobic borders of the cartridge.

Parallel versus Sequential Chemical Reactions: In a parallel embodiment, a different analyte reaction product is generated in the reaction chamber on each layer of the cartridge. In a sequential embodiment, different reaction products are produced sequentially along the length of the same layer. For example, more than one reaction chamber and/or multiple reactions occur sequentially from the middle section of the cartridge towards the front end to produce more than one analyte reaction product and/or the final quantified analyte reaction product. For example, each analyte reaction product can serve as a reactant or reagent in a subsequent reaction downstream on the cartridge.

In an embodiment, the detection mechanism comprises three parallel electrodes (e.g. silver electrodes) with two sections of paper cartridge comprising analyte reaction products residing between the electrodes; or surrounding the right and left side of each electrode.

In one exemplary embodiment, the cartridge may test concurrently for hemoglobin concentration and hematocrit level.

In another exemplary embodiment, the cartridge may test concurrently for sodium, potassium, and calcium concentrations.

### Microfluidic Kit

Kits for use in the various embodiments of the present disclosure comprise: one or more microfluidic cartridges disclosed herein to isolate and quantify a pre-selected analyte from a patient specimen, and written instructions for use of the kits (or website addresses thereto). The kit may further comprise components to facilitate activating the reaction to produce the analyte (e.g. liquid reagents).

In an embodiment, each kit comprises one type of cartridge to detect and/or quantify one type of analyte. The analyte to be tested determines the structure of the cartridge's filter, and impregnated enzymes and/or reagents.

In another embodiment, the kit comprises one type of cartridge to detect and/or quantify more than one type of analyte, wherein each layer of the cartridge tests for a different analyte using different impregnated enzymes and/or reagents; and/or filters for isolating different specimen components.

In another embodiment, each kit comprises more than one type of cartridge to detect and/or quantify more than one type of analyte. For example, one kit may comprise various types of cartridges, wherein each cartridge is used to test for a different type of analyte within a related class, such as for the presence of different types of tumor markers that are associated with the same type of cancer (e.g. breast).

The kits may further comprise a reader device, such as an potentiostat reader device, to detect and quantify the analyte concentration or data, and to electronically transmit the results to one or more electronic computing devices.

In one embodiment, the potentiostat reader device comprises a potentiostat electrical circuit and the detection mechanism of the cartridge comprises three parallel electrodes.

### Microfluidic Testing System

In one or more embodiments, the microfluidic system comprises all of the hardware and software components required for testing at a point-of-care facility, such as one located in a doctor's office. The system comprises: one or more (i.e. a plurality of) disposable microfluidic cartridge devices comprising paper, plastic, and/or cellulose based sensor strips; a microfluidic cartridge reader device that is compatible with the cartridges to compute a test result comprising the amount or concentration of one or more analytes; at least one electronic computing system with software for automatedly receiving and storing the patient's test results from the reader device; a means for scanning and transmitting a patient barcode or QR code associated with a patient specimen; and a wired or wireless network for electronic transmission between the system reader device and electronic computing systems. In an embodiment, the reader device is a potentiostat reader device, and the cartridge front end comprise a plurality of parallel electrodes.

In an embodiment the electronic computing device with software installed on or accessible via the network, is one or more (i.e. a plurality) of electronic computing devices comprising a non-transitory computer readable storage medium comprising instructions, wherein the instructions are operable to enable the device to perform a procedure of receiving an electronic communication from the potentiostat reader device comprising analyte test results and storing the results in a file on the device memory, such as in the patient's medical records file. For example, a patient's smartphone may comprise a mobile app of the present embodiment installed thereon for receiving analyte concentrations; and/or a doctor's office computers may have software of the present disclosure installed or accessible via a network for storing the test results in a patient's electronic medical records.

### General Method of Use of System

The general method of use of the system comprises the steps of: the patient is identified (e.g. using a barcode or QR scanner), and then a fluid specimen is taken from the patient and dropped onto the near the rear end of microfluidic cartridge. The specimen comprises one or more of: blood, saliva, cerebrospinal fluid, joint fluid, urine and tears.

The specimen then travels, via wicking, in a linear manner down the middle of the microfluidic cartridge through a filter, a reaction chamber and then to a detection mechanism. At the filter, pre-selected unwanted specimen components or constituents are filtered out, especially if they will impede the test results. Different cartridges that are used to test for different analytes may filter out the desired specimen component that comprises the desired analyte.

The filtered specimen then travels down the center of the cartridge, and along a straight hydrophilic microfluidic channel extending from the filter to a reaction chamber and from the reaction chamber to a detector mechanism (e.g. three electrodes surrounded by analyte) located on the cartridge top end. At the reaction chamber, impregnated enzymes and reagents chemically react with the filtered components to produce a desired analyte reaction product. The analyte reaction product then continues to travel down the hydrophilic microfluidic channel to the detection mechanism at the cartridge device's front end.

The concentration of the analyte product is then determined by the detection mechanism, which may further comprise use of another reader device, such as the exemplified potentiostat reader device into which the cartridge device front end is inserted, or otherwise makes contact with an electrical connector to the potentiostat circuit, such as one comprising a three pencil lead extending out from the reader device. While the connector is reusable, the only disposable parts are the pencil lead, thus this embodiment reduces the cost of electrode chip significantly. The cartridge and the potentiostat reader device together utilize electro-chemistry and electro-analytics to determine the concentrations of the analyte reaction products by methods well known in the art.

In the exemplified embodiment, the potentiostat reader device comprises a potentiostat circuit that the microfluidic device is inserted into, and the detection mechanism on the cartridge comprises a reference, a working and a counter electrode surrounded by the analyte embedded in the paper. The amount and/or concentration of each analyte is calculated by the central processing unit of the potentiostat reader device, or a computer that the reader device transmits the data to, from the voltage or current detected by the potentiostat circuit (e.g. looking up on standardized curves-graphs of known analyte reaction product concentrations versus voltage), which is then displayed on the potentiostat reader device screen, stored in the memory of the reader device and/or user device, and/or transmitted wirelessly to one or more electronic computing devices for storage in a patient's medical record.

### BRIEF DESCRIPTION OF THE DRAWINGS

The invention and its different aspects and advantages will be better understood from the following detailed description of preferred embodiments of the invention with reference to the following drawing:

FIG. 1 is an illustration of the components of the system and the sequence of steps for their use (from left to right), which is further shown in FIG. 6.

FIG. 2 is an illustration of an potentiostat reader device of the present disclosure with the microfluidic cartridge being inserted into it.

FIG. 3 is an illustration of an exemplification of the microfluidic cartridge device 2 comprising a sensor strip with three assay layers for testing for three different analytes simultaneously (one per each of layer 7a, 7b, and 7c) and a backing strip 8.

FIG. 4 is an illustration of the direction of flow of the specimen from the rear to the front of the cartridge, and the reactions steps of the specimen as it is converted into the analyte reaction product.

FIG. 5 is an illustration of the structural components of each layer of the microfluidic cartridge device.

FIG. 6 is a flowchart of steps in an exemplary method of use of the microfluidic system for testing an analyte produced from a patient's blood specimen and wirelessly transmitting the test results to electronic computing devices.

FIG. 7 is a block diagram of an exemplary reader device comprising a potentiostat within a main circuit board, connected to a Wi-Fi module, that transmits analyte data to a user's electronic computing device with MATLAB® software installed thereon to analyze the data.

FIG. 8, is an electrical schematic diagram of the main circuit board within the reader device of FIG. 7.

FIG. 9 illustrates a graph of micro amperes (y-axis) versus micro volts (x-axis) for a cyclic voltammetry experiment testing a capsaicin solution of 50 micro moles per liter using the reader device of FIGS. 7 and 8 with the cartridge of FIGS. 1-5, which showed a peak current at 100 micro amperes.

FIG. 10 illustrates a linear graph of peak currents in micro amperes (y-axis) versus micro volts (x-axis) of 4 capsaicin solutions at concentrations of 50, 100, 200, 300 micro moles per liter.

### DETAILED DESCRIPTION OF EXEMPLARY EMBODIMENTS

### Glossary of Terms

As used herein, the term “Analyte” or “Analyte Reaction Product” refers to the proteins, polynucleotides (DNA), ribonucleotides (RNA) and/or bio-chemicals that are derived from the patient specimen and quantified by the potentiostat reader device 1. The analyte comprises a reaction product resulting from a chemical reaction that occurs within the cartridge reaction chamber between a filtered patient specimen and impregnate enzymes and/or reagents specifically designed for producing a desired analyte. By way of non-limiting examples, the analyte reaction product comprises one or more of: hemoglobin, platelets, sodium, potassium, creatinine, urea, lactic acid, cholesterol, low density lipoprotein, high density lipoprotein, triglycerides, testosterone, cortisol, prostate specific antigens, and tumor markers.

As used herein, the term “Electronic Computing Device” refers to any electronic computing device comprising a central processing unit (CPU), memory, and with network connectivity (Internet or wired), such as: a hospital or a doctor's office computer; and a patient's laptops, desktops, tablets, iPads, smartphones, cell phones, personal digital assistant devices, and the like. The reader device may also comprise an electronic computing device with a CPU, memory, and the ability to wirelessly transmit analyte data to one or more patient's or doctor's electronic computing devices.

As used herein, the term “Software” refers to computer program instructions, such as a mobile application or software program adapted for execution by a hardware element, such as a processor or CPU, wherein the instruction comprise commands that when executed cause the processor to perform a corresponding set of commands. The software may be written or coded using a programming language, and stored using any type of non-transitory computer-readable media or machine-readable media well known in the art.

Examples of software (and modules) in the various embodiments of present disclosure comprise any software components, programs, applications, computer programs, application programs, system programs, machine programs, and operating system software that the reader device 1 or the user electronic computing device 20 (patient's, doctor's, hospital's, etc.) utilizes to: detect and/or quantify analyte amounts within a patient specimen; and/or store the analyte data within an electronic medical record; and/or use the analyte data to automatedly diagnose a patient's medical condition; and/or use the analyte data to automatedly prescribe a course of treatment.

As used herein, the term “Module” refers to a portion of a computer program or software, and/or hardware that carries out a specific function and may be used alone or combined with other modules of the same program. The module or modules may comprise a mobile application. In a one embodiment, a native application is installed on a user device, wherein it is downloaded automatically from the Internet. It may be written in a language to run on a variety of different types of devices; or it may be written in a device-specific computer programming language for a specific type of device. In another embodiment, a web application resides on the system server and is accessed via the Internet. It performs basically all the same tasks as a native application, usually by downloading part of the application to the device for local processing each time it is used. The web application software is written as Web pages in HTML and CSS or other language serving the same purpose, with the interactive parts in JavaScript or other compatible language. Or the application can comprise a widget as a packaged/downloadable/installable web application; making it more like a traditional application than a web application; but like a web application it uses HTML/CSS/JavaScript and access to the Internet. The module may further or alternatively comprise hardware (e.g. Bluetooth® chip or other Wi-Fi circuits) for wireless transmissions between the potentiostat reader device and a user electronic computing device.

As used herein, the term “non-transitory machine-readable storage medium”, refers to any mechanism that can store information in a form accessible by a machine or device (e.g. a computer, network device, a smartphone, a personal digital assistant (PDA), or any other device with one or more processors, etc.). For example, a machine-accessible storage medium includes tangible recordable/non-recordable media (e.g., read-only memory (ROM) chip; random access memory (RAM) device; magnetic disk storage media; optical storage media; flash memory devices; etc.), etc.

As used herein, the terms “Processing,” “Computing,” “Calculating,” “Determining,” “Analyzing”, or the like, may refer to operation(s) and/or process(es) of a computer, a computing platform, a computing system, a computer central processing unit (CPU), or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

### Microfluidic System

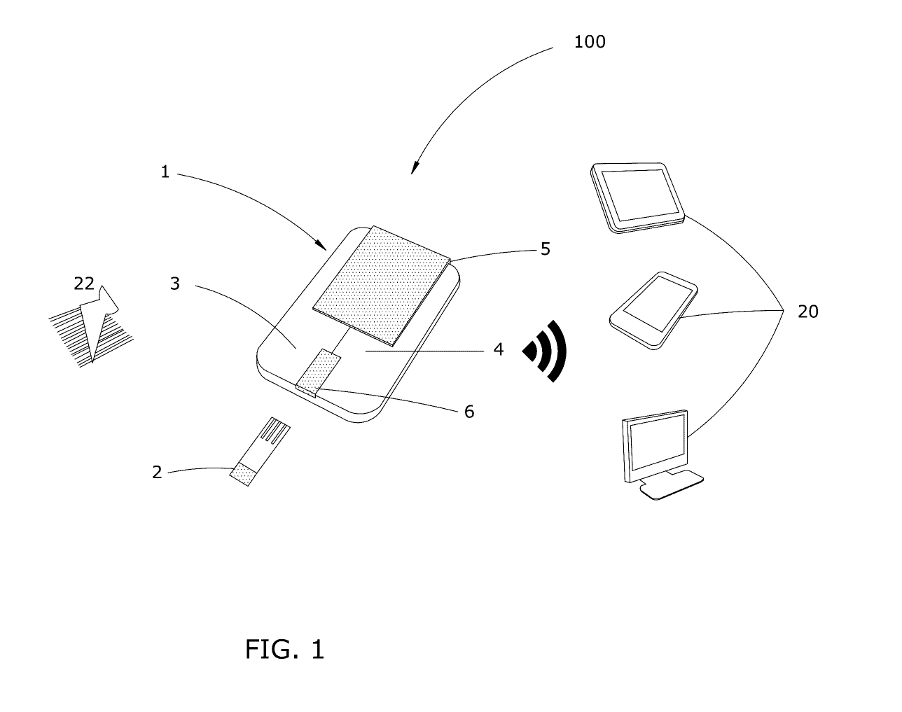

FIG. 1 is an illustration of the microfluidic system 100 and the process of the various embodiments herein, comprising: an Ark Health™ EVA potentiostat reader device 1; a microfluidic device comprising cartridge-sensor strip 2 with one or more testing layers; and further comprising one or more user electronic computing devices 20 for receiving electronic transmissions comprising the test results or data from device 1.

The system of the present embodiments also comprises a wired and/or wireless network for transmitting the electronic communications, such as the Internet or via a variety of methods such as a phone modem, wireless (cellular, satellite, microwave, infrared, radio, etc.) network, Local Area Network (LAN), Wide Area Network (WAN). In the illustrated embodiment, the potentiostat reader device 1 transmits an electronic communication wirelessly using network 4 that is received at a plurality of electronic computing devices 20.

The electronic computing devices 20 may comprise portable electronic computing devices, such as laptops, desktops, tablets, iPads, smartphones, cell phones, personal digital assistant devices, and the like. Device 20 may also comprise non-portable computer systems, such as a hospital or medical clinic computer system. Computing devices or system 20 further comprise one or more of: a central processing unit (CPU); a memory for storing patients' medical records comprising the test results from the potentiostat reader device 1; modules with computer instructions installed on the devices 20 or accessible via a network, for carrying out the steps of the various embodiments disclosed herein, such as software (e.g. MATLAB®) or mobile applications installed on devices 20; and a means for receiving and transmitting electronic communications, such as internet connectivity, cellular service, etc.

The system may further comprise a device 22 for linking patient identification information to the patient specimen, such as a barcode or a quick response code (QR code) reader well known in the art. For example, device 22 may comprise a user electronic computing device with software installed thereon for scanning QR codes (e.g. a smartphone with QR code mobile app). In an embodiment, the coded information would comprise a patient identification code and the one or more analytes being tested for in the specimen. The code is also co-located with a patient's specimen, such as by a being printed on a label that is located on the outside of the specimen container or vial. The coded information may also be electronically transmitted via wired or wireless means to the reader device 1 and/or one or more electronic computing devices 20 (patient's, doctor's, etc.), such as before the specimen is applied to the cartridge 2.

### Potentiostat Reader Device

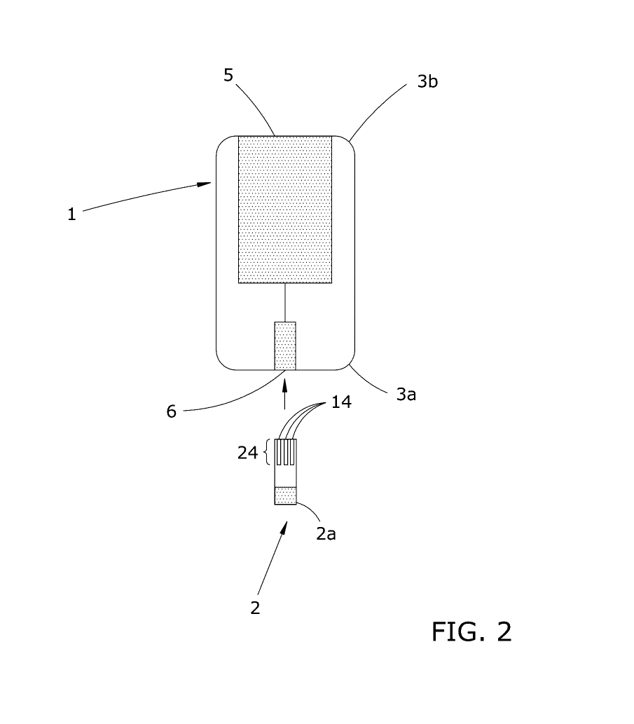

Referring to FIGS. 1 and 2, the exemplified hand-held, portable, potentiostat reader device 1 comprises: a thin rectangular shaped housing 3 with a rear end 3a; a front end 3b; a top flat surface with a screen 5 to display the quantity (e.g. concentration) of one or more analytes; and a bottom flat surface (not shown).

The potentiostat reader device 1 further comprises a screen 5 on the top flat surface, and extending from the front end 3b to about the middle of the top surface. Screen 5 displays the test results, such as the amount or the concentration of the tested analyte (e.g. “134 mmol/L”). Another analyte would be displayed concurrently beneath this, such as screen 5 would display:
Sodium 134 mmol/L
Potassium 4 mmol/L

The top flat surface, rear end 3a further comprises a rectangular shaped slot 6. When the front end of the microfluidic cartridge 2 is inserted into slot 6, the potentiostat reader device 1 calculates the voltage potential or currents of the analytes and electrodes within cartridge 2 using methods of cyclic voltammetry well known in the art.

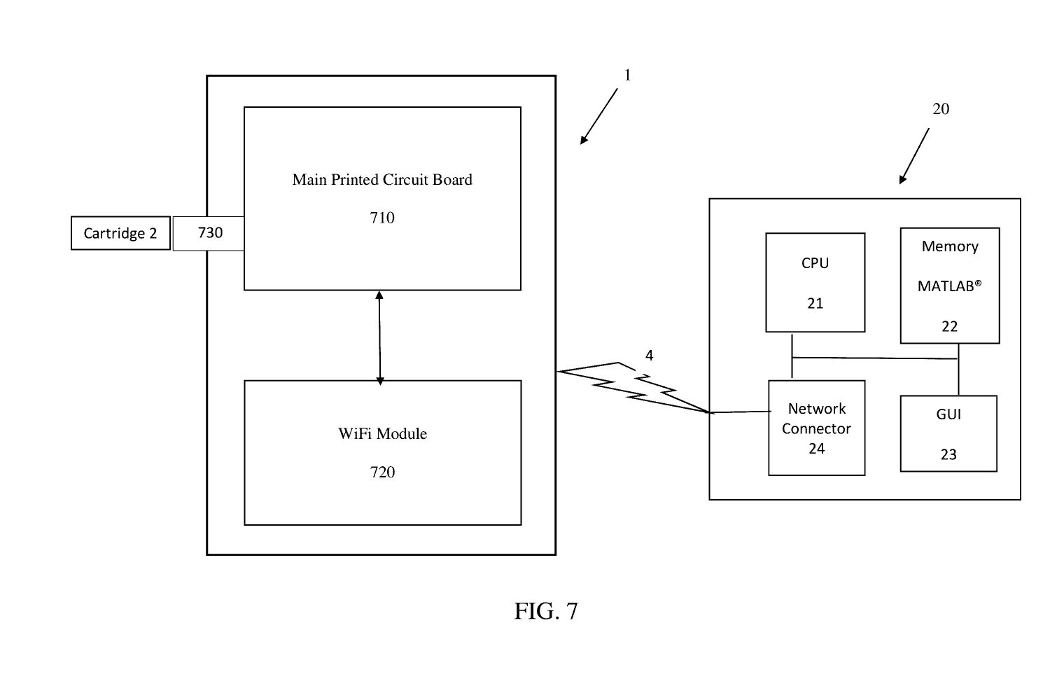

In other embodiments, the slot 6 may reside on other areas of the reader device 1, such as on the left or right side. Or, in lieu of a slot 6 to insert the cartridge 2 into, an electrode connector to the reader device's potentiostat may extend from out of the device to connect with the parallel electrodes 14 on the cartridge 2 (e.g. see FIG. 7, connector 730).

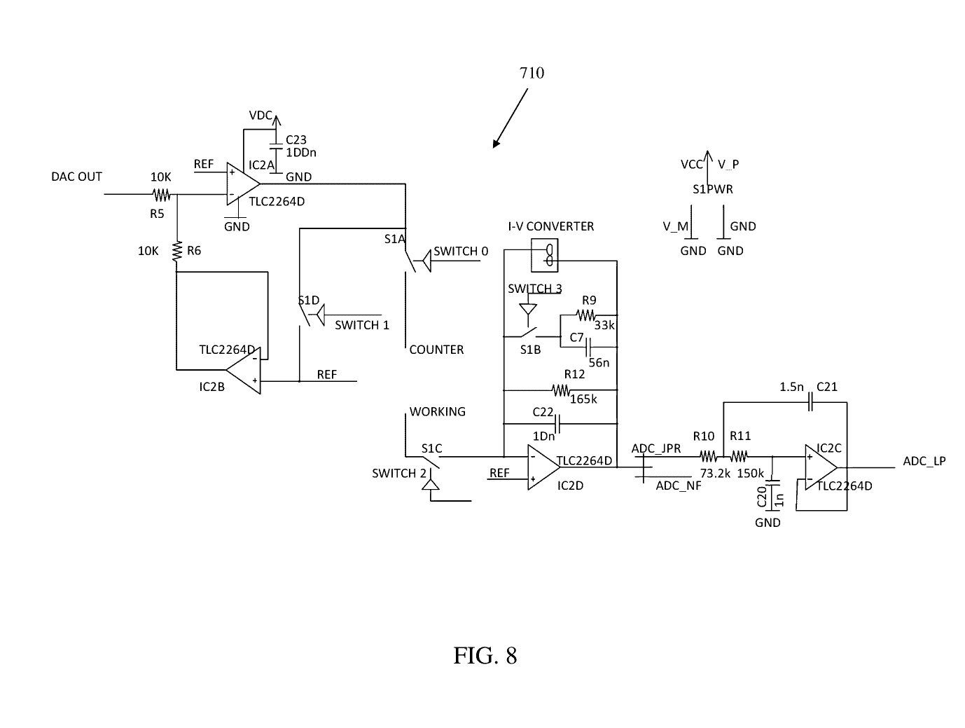

The internal components of the potentiostat reader device 1 within housing 3 further comprise: electrical circuitry (e.g. FIG. 8) of the potentiostat for reading the voltage potentials across the three electrodes 14 of cartridge 2; a central processing unit (CPU) for calculating the amount and/or concentration of the analyte(s) from the voltage potential reading, or for processing data for analysis by device 20; a memory for storing the test results; and internal electronic components for wired and/or wireless transmission of the test results to one or more electronic computing devices 20.

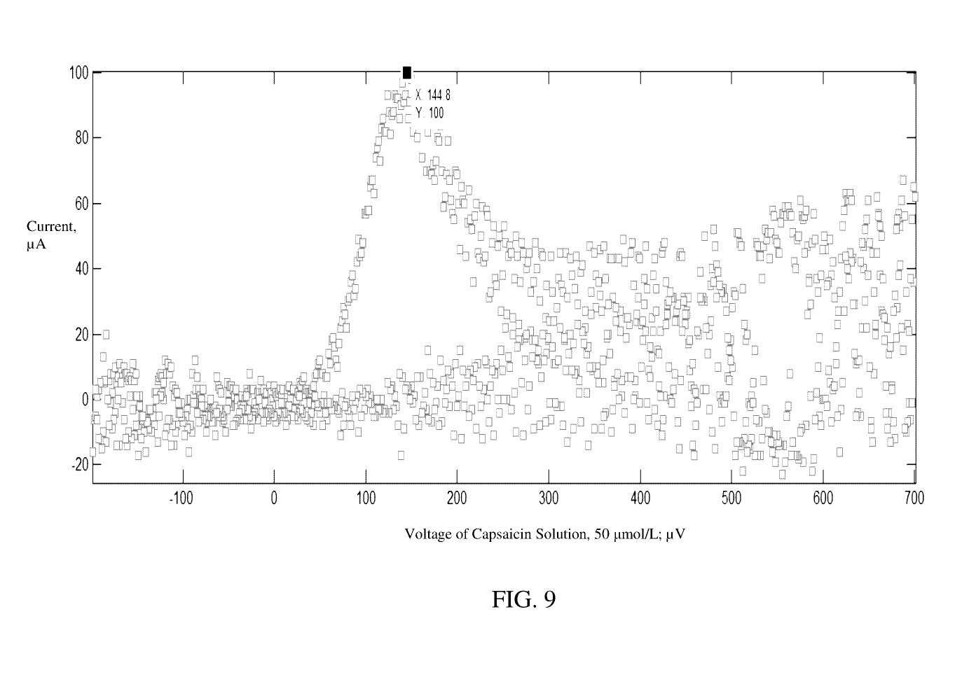

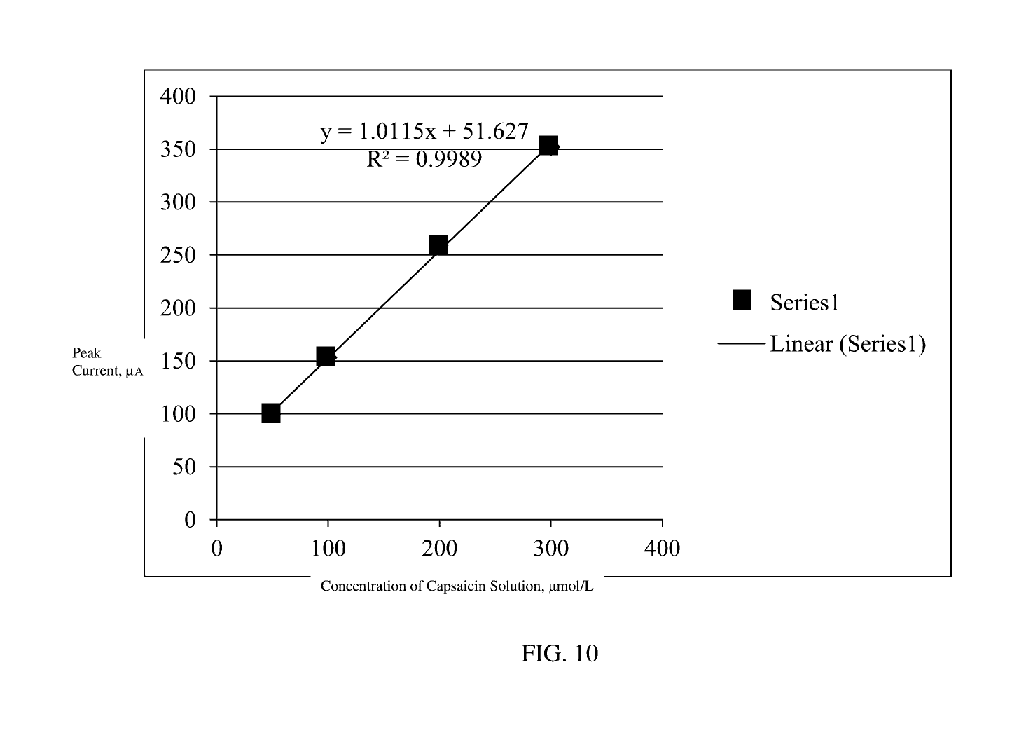

FIG. 7 is a block diagram of another exemplary microfluidic system comprising a cartridge 2 connected to a reader device 1 comprising a potentiostat within a main circuit board 710 (see FIG. 8); connected to a Wi-Fi module 720 that wirelessly transmits analyte data to a user's electronic computing device 20 with MATLAB® software installed within the device's memory 22 to analyze the data and generate the graphs (see FIGS. 9 and 10). The potentiostat circuit 710 collects the signal data generated when the cartridge 2 front end electrodes 14 contact the external electrode connector 730, and the potential across the connected electrodes is continuously measured.

The Wi-Fi module 720 receives the testing result from the main board 710 using a universal asynchronous receiver/transmitter (UART), and it converts the testing results into a Wi-Fi signal. As long as the user electronic computing device 20 with the network connector 24 is in the same network 4 as the Wi-Fi module 720, then the MATLAB® graphical user interface (GUI) 23 and CPU 21 will be able to receive the testing results, and perform the analysis accordingly to generate one or more graphs of the analytes voltage versus the analytes current per cyclic voltammetry and amperometry methods well known in the art (see FIGS. 9 and 10). From these graphs, the central processing unit 21 via the MATLAB® GUI 23 is able to determine the amounts or concentrations of the analytes, which is displayed on the device 20 and/or on the reader device 1.

### Disposable Microfluidic Cartridge Device

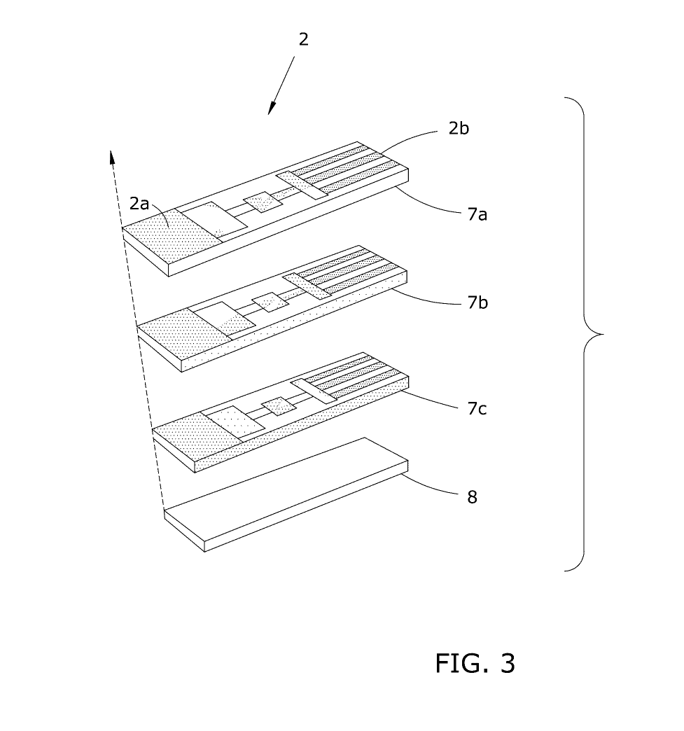

Referring to FIGS. 3 through 5, the exemplified disposable microfluidic cartridge device 2 is thin, rectangular shaped with a rear end 2a and a front end 2b. The front end 2b is inserted into the reader device 1, or otherwise placed in contact with the circuit of FIG. 7, which measures the amount or concentration of one or more analytes on the front end 2b.

The micro-fluidic cartridge device 2 is primarily a multiplexed sensor strip comprising the components of: 1) a backing strip 8 that provides structural support to the assay layer(s) 7; and, 2) one or more assay layers 7 stacked vertically upon the backing strip 8. The backing strip and each assay layer is the same width and length.

FIG. 3 illustrates an example of a multi-layered microfluidic cartridge 2 comprising three assay layers (7a, 7b, and 7c). Each layer can produce a different analyte, or analyte reaction product, from the same patient specimen dropped onto cartridge 2. The potentiostat reader device 1, or user device 20, will then calculate and display the concentration of each of the analytes, concurrently or sequentially.

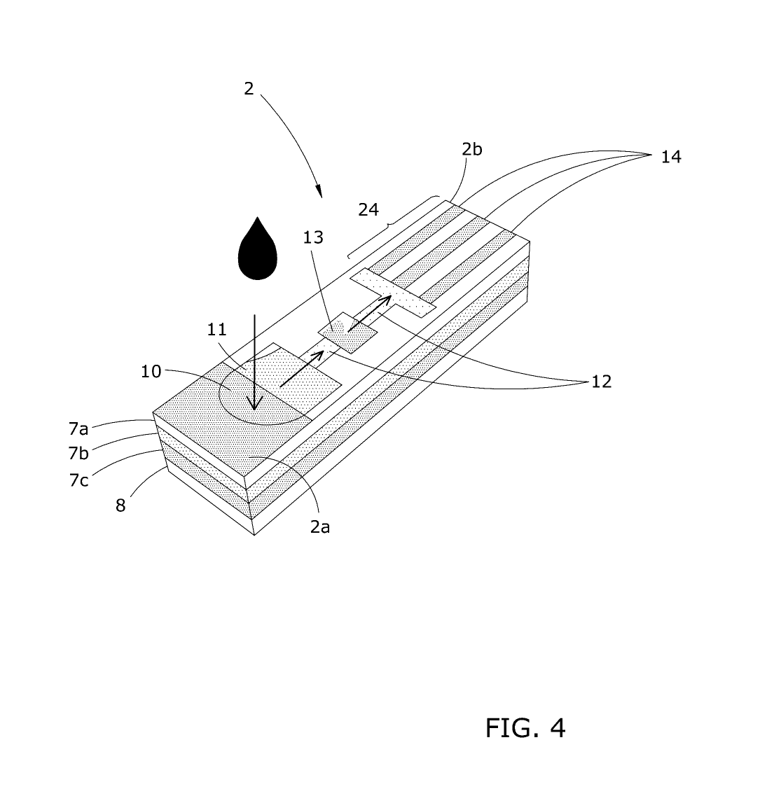

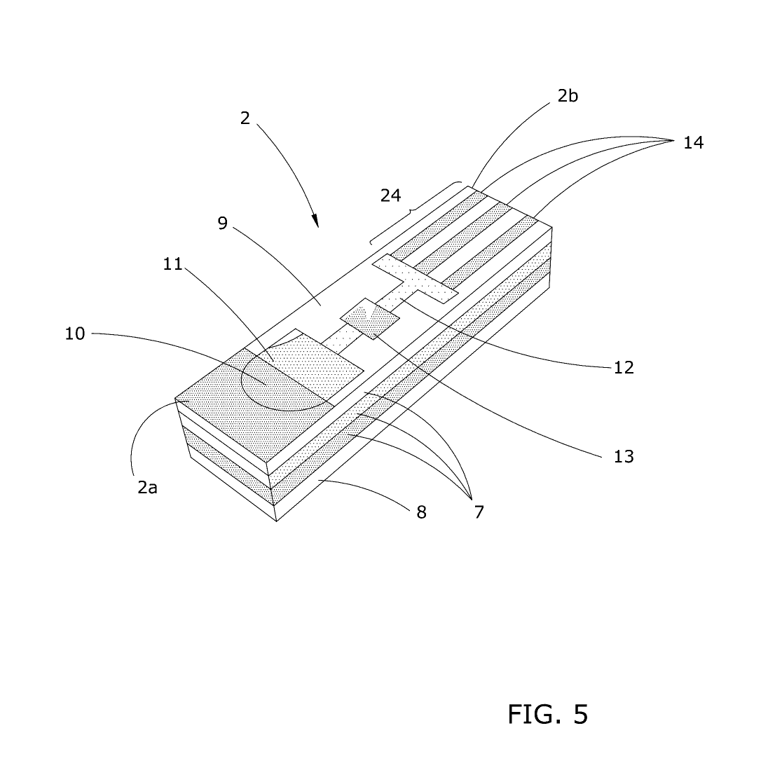

As illustrated in FIGS. 4 and 5, each assay layer 7 further comprises from the rear end 2a to the front end 2b: 1) a drop zone 10 for receiving a patient specimen from a liquid droplet placed by a clinician; 2) a filter 11 that removes contaminates (i.e. undesirable specimen constituents or components), and/or isolates assay reactants; 3) a straight hydrophilic microfluidic channel 12 extending from the filter 11 to the rear end of the reaction chamber 13, and from the front end of the reaction chamber 13 to a detection mechanism 24 comprising in an embodiment three electrodes 14; 4) a reaction chamber 13 comprising a reagent system with impregnated enzymes and reagents to chemically react with the filtered components to produce a specific analyte; and, 5) a detector mechanism 24 for detecting and quantifying the amount or concentration of one or more analytes. The device 2 may further comprise a base layer 8, or a hydrophobic area 9, made from cellulose or plastic material, while other sections or areas of the assay layer 7 (e.g. 10-13, and 24) are made from paper or other disposable material that is able to wick the specimen and its filtered components from the rear end 2a to the front end 2b.

Referring to FIG. 4: the general direction of the specimen's progress is illustrated. After a droplet of specimen is deposited onto zone 10, a portion of the specimen proceeds through the filter 11, and down microfluidic channels 12 (solid arrows) in the most superficial assay layer 7a, interacting in the reaction chamber 13 with chemical reagents impregnated in this layer, and towards the detector mechanism 24 comprising three parallel electrodes 14 interspersed between analyte reaction products. The remainder of the specimen falls through the drop-zone 10 (vertical arrow) to one or more deeper assay layers (e.g. 7b, 7c).

At these deeper assay layers, the specimen travels away from the drop-zone 10 (horizontal arrows) down the micro-fluidic channel 12 like in the most superficial layer 7a. In an embodiment, as the filtered specimen reaches the reaction chamber 13 in each underlying layer (e.g. 7b, 7c), a different analyte reaction product is produced. Each analyte reaction product subsequently moves to the detector mechanism 24 located on the front end 2b of the cartridge 2. The reader device 1 then computes the concentration of each analyte on each layer, and displays the results concurrently or sequentially on the screen 5 of the reader device 1, and/or on the user device 20.

The filter 11 removes components or constituents and/or ions from the specimen that would interfere with the assay. These unwanted filtered specimen constituents may drain vertically downward towards layer 7b, 7c, and/or backing strip 8. The filter 11 may also lyse cells to release the assay reactants required for the assay that occurs within the reaction chamber 13. The structure and material components of filter 11 may also be specifically designed per the type of specimen fluid and the desired analyte, such as cholesterol, which requires additional processing steps to separate it from blood.

The reaction chamber 13 is where the chemical assay is performed for producing a specific analyte that is then quantified using the detection mechanism 24. The assay reaction product(s) comprises the analyte produced on each layer (e.g. layer 7a, 7b, and 7c) of the cartridge 2. By way of non-limiting examples, the analytes comprise one or more of: hemoglobin, platelets, sodium, potassium, creatinine, urea, lactic acid, cholesterol, low density lipoprotein, high density lipoprotein, triglycerides, testosterone, cortisol, prostate specific antigens, and tumor markers.

In addition to the generation of different analytes in parallel layers 7a, 7b, and 7c, different reaction products may be produced sequentially along the length of the same layer. For example, more than one reaction chamber 13 and/or reactions may occur sequentially from the middle section of the cartridge towards the front end 2b to produce more than one analyte reaction product and/or the final quantified analyte reaction product. For example, each analyte reaction product can serve as a reactant or reagent in a subsequent reaction downstream on the cartridge strip 2 per each layer 7a, 7b, and 7c.

Detection Mechanism: in the exemplified embodiment where the microfluidic device 2 is used in an electro-chemical assay, the detection mechanism 24 comprises in one embodiment three parallel electrodes 14 (e.g. silver electrodes) at the front end 2b of the cartridge 2 interspersed around and between two strips of analyte reaction products. The three electrodes function as a reference, working and counter electrode via methods well known in the art of cyclic voltammetry. The voltage potential of the electrodes and analyte is measured by a potentiostat electrical circuit housed within a reader device 1 that the microfluidic device 2 front end 2b is inserted into the reader slot 6.

Based on the disclosure herein, one of skill in the art could readily make similar microfluidic cartridges 2 that are able to produce equivalent analytes reaction product, while using other detection mechanisms 24, such as those comprising colorimetric or spectrophotometric technologies. For example, the microfluidic cartridge 2 for use with other types of reader devices 1 would comprise: 1) a drop zone 10; 2) a filter 11; 3) a straight hydrophilic microfluidic channel 12 extending from the filter 11 to the rear end of the reaction chamber 13, and from the front end of the reaction chamber 13 to a different type of detection mechanism 24; and 4) a reaction chamber 13 comprising a reagent system with impregnated enzymes and reagents to chemically react with the filtered components electro-chemical analytics.

### Exemplification: Method of Electro-Chemical Analysis

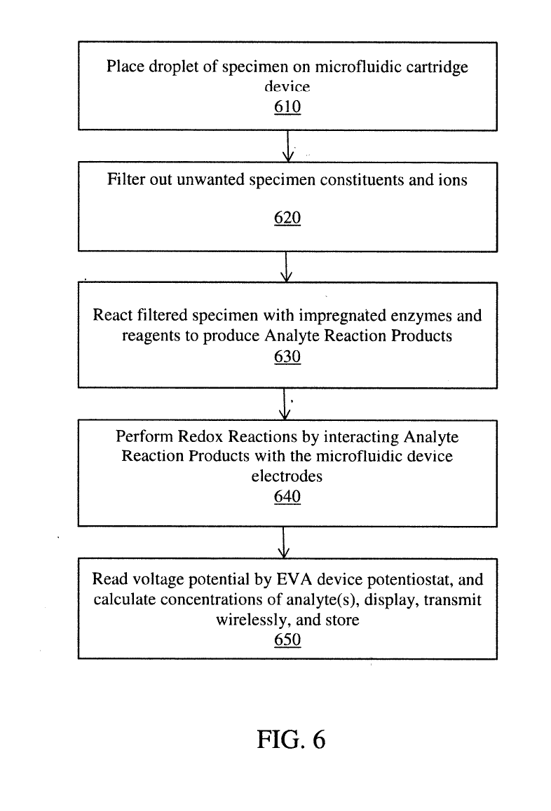

FIG. 6 is a flowchart of steps in an exemplary method of use of the disposable microfluidic device and the system for testing the concentration of an analyte produced from a patient's blood specimen, and transmitting the test results to remote computers for storage within a patient's electronic medical record.

In step 610, the clinician scans a barcode (or QR code, or the like) that contains the patient identification information. The code may further comprise the one or more analytes being tested for. Referring to FIGS. 3-5, the clinician then takes a sample of a patient specimen (e.g. blood), and places a droplet of it near the rear end 2a of the disposable microfluidic cartridge 2 within the drop-zone 10.

In step 620, the specimen travels towards the filter 11 (e.g. via wicking), which adjoins the front end of the drop zone 10. Filter 11 can: 1) remove component or constituents and/or ions from the specimen that would interfere with the assay that occurs in the reaction chamber 13; and/or 2) lyse cells to release the analytes required for the assay. The filtered specimen (otherwise known as the “target analyte”) then travels down hydrophilic microfluidic channels 12, and towards the reaction chamber 13 that is located in the middle of the cartridge 2.

In step 630, the self-contained reaction chamber 13 comprises enzymes and reagents impregnated into the layer 7a, and designed to isolate and/or produce a selected analyte reaction product from the filtered specimen. It is noted that more than one reaction chamber 13 and/or reactions may occur sequentially from the middle section of the cartridge towards the front end of the cartridge, so as to produce more than one analyte reaction product and/or the final quantified analyte reaction product. For example, each analyte reaction product can serve as a reactant or reagent in a subsequent reaction downstream on the cartridge strip 2. And more than one analyte reaction product can be produced in parallel by having multiple layers 7a, 7b, 7c with each layer comprising different enzymes and reagents to produce different analyte reaction products.

In step 640, the analyte reaction product(s) travels down the hydrophilic microfluidic channels 12 from the reaction chamber 13 towards the cartridge device top end 2b where detection mechanism 24 resides. In the exemplified embodiments illustrated in FIGS. 1-5, the detection mechanism comprises three parallel electrodes 14 with analyte reaction products lying between or surrounding the electrodes 14. Redox reactions are performed using methods well known in the art by interacting the analyte reaction products with the microfluidic reader device 1 external electrode connector 730 (FIG. 7) or internal connector within slot 6 (FIGS. 1-2). In an embodiment, while the potentiostat reader device 1 is on and a constant current is applied while cycling the voltage (−990 mV to 990 mV, using a frequency from 1 to 1000 Hz).

In step 650, the cartridge device top end 2b is inserted in the potentiostat reader device slot, and the electro-analytical methods (e.g. cyclic voltammetry, potentiometry, amperometry, etc.) from the redox reactions are measured by the potentiostat circuit housed within the reader device 1. The data is wirelessly transmitted to a user electronic computing device 20, upon which the central processing unit (CPU) 21 of the device 20 correlates the potentials or peak current with known analyte reaction product concentrations on a standard curve for a particular analyte reaction product (i.e. standard curves of known analyte reaction product concentrations versus voltage or peak current).

The computed concentrations of each analyte reaction product is then displayed on the user interface 5 of the reader device 1 and the device 20, and transmitted via the internet to one or more computer systems and electronic computing devices 20 for storage in the patient's medical record and/or for displaying on the user's device (e.g. an automatedly email to the patient displaying their test results).

### EXEMPLIFICATIONS

Per the methods well known in the art for cyclic voltammetry, and referring again to FIG. 7, which is a block diagram of an exemplary reader device 1 comprising a potentiostat within a main circuit board 710, that is connected to a Wi-Fi module 720 enabled to wirelessly transmit (e.g. via Bluetooth® or other wireless means well known in the art) analyte data to a user's electronic computing device 20. Device 20 may further have software, e.g. MATLAB®, installed within the device's memory 22 to analyze the data and generate the graphs (e.g. FIGS. 9-10). The potentiostat circuit 710 collects the signal data generated when the cartridge 2 front end electrodes 14 contact the potentiostat circuit, such as with the external electrode connector 730. Then the peak current for a plurality of different concentrations of an analyte is determined (previously as a standard control) and plotted in a linear graph.

FIG. 8, is an electrical schematic diagram of the main circuit board 710 within the reader device of FIG. 7. The main controller board 710 consists of analog circuitry that connects to the reference, working and counter electrodes. The analog circuitry itself consists of a system of resistors, capacitors, switches and operational amplifiers (as illustrated in FIG. 8). The system is carefully grounded to ensure signal integrity and to maximize noise suppression. When analytes undergo biochemical reduction-oxidation reactions on the cartridge strip 2, their intermediates or products may carry positive or negative charges and interact with the reference, working and counter electrodes. This may occur in various embodiments. The reference, working and counter electrodes from the main controller board 710 may extend directly into the part of cartridge strip 2 where the redox reaction intermediates or products are concentrated. In another embodiment, cartridge strip 2 itself may contain corresponding reference, working and counter electrodes that connect to the respective electrodes on the analog circuit. In yet another embodiment, an intermediate connector 730 may bridge the electrical connection between cartridge strip 2 and main controller board 710. In any one of these embodiments, the resultant voltage or currents are converted into electrical signals on the main controller board 710 which are passed onto the Wi-Fi module 720 and the user device 20 for further analysis.

Per the methods of cyclic voltammetry, the reader device housing the electrical potentiostat circuit 710 of FIG. 8, applies a potential between the working electrode and the reference electrode while the current is measured between the working electrode and the counter electrode. After a set potential is reached, then the working electrode's potential is ramped in the opposite direction to return to the initial potential. In an embodiment, the main controller board 710 has a cyclic potential with range from −990 mV to 990 mV, and frequency from 1 to 1000 Hz.

The data is plotted as current (i) in units of micro amperes (μA) on the y-axis, versus voltage in millivolts (mV) on the x-axis for a specific concentration of the analyte. FIG. 9 is a graph from an exemplary cyclic voltammetry displayed on the user electronic computing device GUI 23 when tested on a capsaicin solution of 50 micro moles per liter (mM/L). The y-axis demonstrates the peak current of 100 micro Amperes (the highest peak), for varying voltages (x-axis, with units in micro Volts-μV).

The cyclic voltammetry is repeated for multiple concentrations of capsaicin: 100, 200, 300, and 500 micro moles per liter, and the peak current at each concentration is determined. The peak currents are then plotted (y-axis) versus the capsaicin concentration (x-axis). FIG. 10 is a graph of peak currents obtained by the reader device 1 (y-axis, in micro-amperes), for varying concentrations of capsaicin solution (x-axis, in micro moles per liter). This demonstrates the linearity of the reader device's performance. The R2 value (i.e. coefficient of determination) close to 1 indicates a good fit between the experimental data points and the line of the graph.

This process of cyclic voltammetry is repeated for each analyte of a known concentration that will be tested on the cartridge reader 2, and a digital linear graph of peak currents versus analyte concentrations (e.g. FIG. 10) is stored in the memory of the reader device 1, and/or the memory of the user electronic computing device 20. These linear graphs are used as a standard-control curve that were generated from the samples of known concentration of analytes. Then when subsequently a patient's specimen is tested, the peak current of each analyte is computed using circuit 710, and the linear graph is used to extract the analyte concentration from the standard curve. The analyte concentration is then displayed on device 20 GUI 23, and may further wirelessly transmitted back to the reader device 1 for display on screen 5.

### CONCLUSION

The techniques introduced herein can be implemented by, for example, programmable circuitry (e.g., one or more microprocessors) programmed with software and/or firmware, or entirely in special-purpose hardwired circuitry, or in a combination of such forms. Software or firmware for use in implementing the techniques introduced here may be stored on a machine-readable storage medium and may be executed by one or more general-purpose or special-purpose programmable microprocessors.

In addition to the above mentioned examples, various other modifications and alterations of the invention may be made without departing from the invention. Accordingly, the above disclosure is not to be considered as limiting, and the appended claims are to be interpreted as encompassing the true spirit and the entire scope of the invention.

The various embodiments are described above with reference to flowchart illustrations and/or block diagrams of methods, apparatus (systems) and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

One or more features or steps of the disclosed embodiments can be implemented using an Application Programming Interface (API). An API can define on or more parameters that are passed between a calling application and other software code (e.g., an operating system, library routine, function) that provides a service, that provides data, or that performs an operation or a computation.

The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API. In some implementations, an API call can report to an application the capabilities of a device running the application, such as input capability, output capability, processing capability, power capability, communications capability, etc.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The aforementioned flowchart and diagrams illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

Furthermore, it is to be understood that the disclosure can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

What is claimed is:

1. A disposable microfluidic cartridge device for detecting and quantifying the amount of an analyte in a fluid specimen, wherein the microfluidic cartridge device comprises:
a. a backing strip providing structural support to one or more assay layers;
b. one or more assay layers stacked vertically upon the backing strip, each layer comprising,
i. a drop zone for receiving a droplet of a patient specimen;
ii. a filter configurable to remove contaminates, and/or to isolate assay reactants;
iii. a straight hydrophilic microfluidic channel extending from the filter to a reaction chamber, and from the reaction chamber to a detector mechanism;
iv. a reaction chamber comprising impregnated enzymes and reagents to chemically react with the assay reactants to produce one or more analyte reaction products; and,
v. a detector mechanism for facilitating a reader device to detect and/or quantify the amount or concentration of the one or more analyte reaction products using electro-analytic techniques.

2. The microfluidic cartridge device of claim 1, wherein the specimen comprises one or more of: blood, saliva, cerebrospinal fluid, joint fluid, urine or tears.

3. The microfluidic cartridge device of claim 1, wherein the assay reaction product(s) comprises on each assay layer one or more of: hemoglobin, platelets, sodium, potassium, creatinine, urea, lactic acid, cholesterol, low density lipoprotein, high density lipoprotein, triglycerides, testosterone, cortisol, prostate specific antigens, and tumor markers.

4. The microfluidic cartridge device of claim 1, wherein the reaction chamber comprises an assay with a reagent system comprising enzymes and reagents specific to producing the analyte reaction product.

5. The microfluidic cartridge device of claim 1, wherein the filter removes components or constituents and/or ions from the specimen that would interfere with the assay.

6. The microfluidic cartridge device of claim 4, wherein the filter lyses cells to release the assay reacts required for the assay that occurs within the reaction chamber.

7. The microfluidic cartridge device of claim 1, wherein the electro-analytic techniques comprise one or more of: cyclic voltammetry, potentiometry, and amperometry.

8. The microfluidic cartridge device of claim 1, wherein the detection mechanism comprises three parallel electrodes and the voltage potential of the electrodes that is measurable by a potentiostat electrical circuit housed within a reader device that the microfluidic cartridge device is connected to.

9. A microfluidic system for detecting and quantifying an analyte reaction product in a fluid sample from a patient's specimen, the system comprising:
a. microfluidic cartridge device configured to conduct an assay to isolate and quantify an analyte reaction product from a patient's specimen, the cartridge device comprising:
i. a backing strip providing structural support to one or more assay layers;
ii. one or more assay layers stacked vertically upon the backing strip, each layer comprising,
a drop zone for receiving a droplet of a patient specimen;
a filter configurable to remove contaminates, and/or to isolate assay reactants;
a straight hydrophilic microfluidic channel extending from the filter to a reaction chamber, and from the reaction chamber to a detector mechanism;
a reaction chamber comprising impregnated enzymes and reagents to chemically react with the assay reactants to produce one or more analyte reaction products; and,
v. a detector mechanism for facilitating a potentiostat reader device in detecting and quantifying the amount or concentration of the one or more analyte reaction products using electro-analytic techniques;
b. a potentiostat reader device housing a potentiostat electrical circuit;
c. one or more electronic computing devices with patient medical records stored in memory and with network connectivity;
d. a computing device for reading a barcode or QR code comprising patient and analyte reaction products identification information and with a network connectivity able to wirelessly transmit the information; and,
e. a network for transmitting the one or more analyte reaction product concentrations and patient identification information to the patient medical record stored on the electronic computing device.

10. The microfluidic system of claim 9, wherein the one or more electronic computing devices further comprises a non-transitory computer readable storage medium comprising instructions, the instructions being operable to enable the electronic computing device to perform a procedure for receiving an electronic communication from the reader device comprising analyte reaction product data and computing the analyte concentration from the data.

11. The microfluidic system of claim 9, wherein the potentiostat reader device further comprises:
a. a potentiostat circuit with a mechanism to connect with a microfluidic cartridge electrodes;
b. a central processing unit for calculating the one or more analyte reaction products amount or concentration, or for collecting data when a voltage or current is applied to the potentiostat circuit;
c. a user interface for displaying the analyte reaction product concentrations;
d. a memory for storing the analyte reaction product concentrations or data; and,
e. electronic components for wired and/or wirelessly transmitting the analyte reaction product concentrations or data and patient identification information to the electronic computing device(s); and,
f. wherein the reader device is configurable to perform electro-analytical methods to facilitate determining the analyte reaction product amount or concentration.

12. The microfluidic system of claim 11, wherein the microfluidic cartridge device detection mechanism comprises three electrodes residing in parallel and surrounded by an analyte reaction product, and the reader device or the electronic computing device further comprises a central processing unit configured to determine the analyte reaction product amounts or concentrations from standardized curves of known analyte reaction product concentrations versus peak currents or voltage.

13. The microfluidic system of claim 11, further comprising a specimen assay kit comprising:
a. one or more disposable microfluidic cartridge devices; and
b. a potentiostat reader device able to perform electro-analytics on the cartridge device.

14. A method of detecting and quantifying one or more analyte reaction products in a patient fluid specimen on a microfluidic cartridge device, the method comprising:
a. placing a droplet of a patient fluid specimen on a disposable, multi-layered microfluidic cartridge device rear end and allowing the droplet to wick to the cartridge device front end;
b. filtering out unwanted specimen constituents and ions, near the cartridge rear end;
c. reacting remaining constituents in a microfluidic cartridge device reaction chamber, located in the cartridge device center, to produce one or more analyte reaction products;
d. detecting and quantifying the concentration of the one or more analyte reaction products utilizing the detection mechanism of the microfluidic cartridge device on the cartridge front end with a potentiostat reader device and using methods of cyclic voltammetry; and,
e. wherein the specimen comprises one or more samples of a patient blood, saliva, cerebrospinal fluid, joint fluid, urine and/or tears.

15. The method of detecting and quantifying an analyte reaction product of claim 14, wherein the microfluidic cartridge device comprises:
a. a backing strip providing structural support to one or more assay layers;
b. one or more assay layers stacked vertically upon the backing strip, each layer comprising,
i. a drop zone for receiving a droplet of a patient specimen;
ii. a filter configurable to remove contaminates, and/or to isolate assay reactants;
iii. a straight hydrophilic microfluidic channel extending from the filter to a reaction chamber, and from the reaction chamber to a detector mechanism;
iv. a reaction chamber comprising impregnated enzymes and reagents to chemically react with the assay reactants to produce one or more analyte reaction products; and,
v. a detector mechanism comprising a reference, working, and counter electrode for facilitating a potentiostat reader device in detecting and quantifying the amount or concentration of the one or more analyte reaction products using electro-analytic techniques.

16. The method of detecting and quantifying an analyte reaction product of claim 14, wherein detecting and quantifying the concentration of the analyte reaction product further comprises inserting the microfluidic cartridge device into a potentiostat reader device comprising,
a. a potentiostat circuit with a mechanism to connect with a microfluidic cartridge electrodes;
b. a central processing unit for calculating the one or more analyte reaction product amounts or concentrations, or for collecting data when a voltage is cycled on the potentiostat circuit;
c. a user interface for displaying the analyte reaction product amount or concentration;
d. a memory for storing the analyte reaction product amount or concentration; and,
e. one or more electronic components for wired and/or wirelessly transmitting the data used to compute the analyte reaction product amount or concentration and a patient identification information to an electronic computing device(s).

17. The method of detecting and quantifying an analyte reaction product of claim 14, further comprising scanning a barcode or QR code comprising a patient identification information associated with the patient specimen, and electronically transmitting the patient identification information to the potentiostat reader device.

18. The method of detecting and quantifying an analyte reaction product of claim 14, wherein reacting remaining constituents comprises chemically reacting enzymes and reagents impregnated into the microfluidic device reaction chamber to produce the analyte reaction products.

19. The method of detecting and quantifying an analyte reaction product of claim 18, wherein the microfluidic cartridge device comprises three electrodes in parallel, and the analyte reaction product reader device comprises a potentiostat electrical circuit to measure the voltage potential of the electrodes and the analyte reaction products, and the reader device CPU or a CPU of a user electronic computing device determines the analyte reaction product concentrations from standardized curves of known analyte reaction product concentrations versus peak currents or voltages.

20. The method of detecting and quantifying an analyte of claim 19, wherein the analyte reaction product comprises one or more of: hemoglobin, platelets, sodium, potassium, creatinine, urea, lactic acid, cholesterol, low density lipoprotein, high density lipoprotein, triglycerides, testosterone, cortisol, prostate specific antigens, and tumor markers.
